(19) United States
(12) Patent Application Publication
(10) Pub. No.: US 2011/0262389 A1
(43) Pub. Date: Oct. 27, 2011

(54) Tumor-derived Biological Antigen Presenting Particles

(76) Inventor: Joseph D. Mosca, Ellicott City, MD (US)

(21) Appl. No.: 13/097,028

(22) Filed: Apr. 28, 2011

Related U.S. Application Data
(63) Continuation of application No. 10/528,082, filed on Mar. 14, 2005, now abandoned.
Application No. 10/528,082 is a continuation-in-part of application No. 12/705,032, filed on Feb. 12, 2010.
Application No. 12/705,032 is a division of application No. 10/486,415, filed on Feb. 6, 2004, now Pat. No. 7,662,608.
Application No. 10/486,415 is a continuation-in-part of application No. 12/538,158, filed on Aug. 9, 2009.
Application No. 12/538,158 is a division of application No. 10/468,047, filed on Aug. 13, 2003, now Pat. No. 7,579,007.
(60) Provisional application No. 60/324,835, filed on Sep. 25, 2001.
(60) Provisional application No. 60/268,066, filed on Feb. 13, 2001.

(51) Int. Cl. A61K 38/20; A61K 39/12; A61P 35/00; A61K 39/395

(52) U.S. Cl. 424/85.2; 424/174.1; 424/204.1

(57) ABSTRACT

Disclosed is a biological anti-tumor approach that delivers specific antigen stimulation in the presence of co-stimulatory signals to the immune system. Tumor cells are engineered such that processed antigens and co-stimulatory molecules are incorporated into virus-like particles capable of modulating immune responses without cellular entry and nucleic acid genomic host cell integration. The invention describes constructing antigen presenting particles that will themselves present tumor antigens in an immunogenic fashion to therapeutically activate anti-tumor immunity within a mammalian host.

This application is a continuation of U.S. patent application Ser. 10/528,082, filed Mar. 14, 2005, which is a national stage entry under 35 U.S.C. §371 of PCT/US04/29671, filed Sep. 11, 2004; and a continuation-in-part of U.S. patent application Ser. No. 12/705,032, filed Feb. 12, 2010, which is a divisional application of Ser. No. 10/486,415, filed Feb. 6, 2004, now U.S. Pat. No. 7,662,608, which is a national state entry of PCT/US02/30197, filed Sep. 24, 2002, and claims priority to U.S. Provisional Patent Application 60/324,835; filed Sep. 25, 2001; and a continuation-in-part of U.S. patent application Ser. No. 12/538,158, filed Aug. 9, 2009, which is a divisional application of Ser. No. 10/486,047, filed Aug. 13, 2003, now U.S. Pat. No. 7,579,007, which is a national state entry of PCT/US02/04157, filed Feb. 12, 2002, which claims priority to U.S. Provisional Patent Application 60/268,066, filed Feb. 13, 2001. Each of the above-identified applications is hereby incorporated by reference as if fully set forth herein.

### FIELD OF THE INVENTION

The present invention relates to the field of immune stimulation in mammalian cells, where biologically generated particles mimic antigen presenting cells by presenting to the host immune system tumor specific antigens in the presence of co-stimulatory molecules leading to immune awareness of in vivo tumor growth.

### BACKGROUND OF THE INVENTION

The single most important risk factor for cancer is age. Because the U.S. population is both growing and aging, the cancer burden in about 50 years from now by applying U.S. Census Bureau population projections to current cancer incidence rates will double. We can anticipate an increase from 1.3 million people in 2000 to 2.6 million people in 2050 diagnosed with cancer and the number of cancer patients age 85 and over is expected to increase four-fold in this same time period. Early diagnosis has led to more effective treatment, but these early detection methods are available for only a limited number of cancers. For many years, the treatment of cancer was primarily focused on surgery, chemotherapy, and radiation. But as researchers learn more about how the body fights disease on its own, therapies are being developed that harness the body's defense system in the fight against cancer. The body's immune defense system is a network of specialized cells. Therapies that use the immune system to fight cancer are biological therapies and the present patent application describes novel technology that can result in anti-tumor responses. The invention relates to a nanotechnology approach by creating particles (1 to 100 nanometers) with novel properties, properties that are normally found associated with cells. This invention application has tremendous potential to meet the present and future demand for therapeutic products for cancer biomedicine.

The enhancement of immune responses in the vaccine setting can be divided mechanistically into three categories: (i) Enhancement of the presentation of antigens to T cells, which implies an increase in peptide-MHC density at the site of activation of T cells; (ii) Enhancement of co-stimulation, which accounts for the fact that T cells require extra co-stimulatory signals—either cell surface-bound molecules such as B7 or soluble molecules such as cytokines—in addition to engagement of the T cell receptor in order to become efficiently activated; (iii) Local elaboration of cytokines that attract and locally activate bone-marrow-derived antigen presenting cells that process and present tumor antigens to T cells. Most of these strategies are based on the engineering of tumor cells, requiring in vitro expansion of primary mammalian tumor explants. This, in addition to the potential hazards associated with cellular gene delivery approaches, present potential hazards in the genetically altered tumors-based enhanced immunogenicity approach. Other approaches may overcome the need to in vivo expansion of tumor tissues, but requires harvesting enough cells from the patient to produce the vaccine, limiting therapy to those where tumor cells could be collected through pheresia or surgical excision of the tumor mass. These cancers include leukemia, lymphoma, multiple myeloma, colon cancer, renal cancer, and ovarian cancer. The current technology as described above is problematic to cancer therapy for the mass population at risk; it is geared towards individual care, not to treat the large numbers of individuals that require treatment.

As an alternative to modifying the tumor cell, several approaches enhance antigen presenting cell (APC) function. Interest has been mainly focused on dendritic cells (DC), which represents the most potent professional APC. Dendritic cells have been shown to allow priming of naïve T cells and to initiate immune responses. They are unique APC that express a high level of MHC peptide complexes (signal 1) along with a collection of co-stimulatory signals (signal 2)—secreted cytokines and membrane-bound accessory molecules. These accessory molecules are representatives of the B7- and TNF-family of molecules. Since qualitative and quantitative aspects of T cell activation are determined by the co-stimulatory signals delivered by APC, much research has focused on dendritic cell associated molecules that regulate T cell biology. Within the B7-family of co-stimulatory molecules, B7-1 and B7-2, has been most widely studied in cancer vaccination, however new members are emerging—B7-DC, a molecule expressed exclusively on dendritic cells demonstrates potent synergistic T cell costimulatory effects in conjugation with B7-1 and B7-2.

The central role of antigen presenting cells in cancer vaccination has led to techniques for growth of dendritic cells (DC) in culture, resulting in ex vivo generated DC vaccines. However, technological problems face DC vaccine use. The prominently recognized issues are the timing of antigen(s) exposure to the antigen presenting cells and the cytokines required to differentiate the antigen presenting cell towards the activated differentiation state. Not withstanding the costly labor-intensive limited availability therapeutic approach associated with DC cell-based therapy, the qualitative and quantitative nature of an immune response depends on the maturation and type of antigen presenting cell that processes and presents antigens to T cells. Although mature dendritic cells are more than 100 times more potent antigen presenting cells than other professional antigen presenting cells, such as B-cells and macrophages in activating naïve T cells in vitro, it is the early differentiating antigen presenting cell, not the mature dendritic cell, that posses the specialized antigen uptake and antigen processing machinery. Establishing the appropriate timing for antigen processing within ex vivo maturation of bone marrow antigen presenting cells have lead to a tripartite approach—lentiviral transduction of hematopoietic stem-progenitor cells, bone marrow transplant followed by infusion of mature post-thymic lymphocytes, and systemic infusion of Flt-3 ligand/anti-CD40—to increase the expression of antigens by dendritic cells in vivo. The potential opportunity to substitute dendritic cell ex vivo maturation/antigen presentation with pre-formed inactivated particles that are engineered biologically in vitro and implanted in vivo to directly interact with T cells to achieve the activated state for anti-tumor responses is the core premise of the present application.

The invention, as described in this application, is geared toward treatment strategies for the mass population at risk for cancer. Instead of cells, the biological particles/carrier approach, relies on particles; the expansion of particles exceeds the expansion of cells by greater than a million-fold since one modified cells can create at least 10E6 particles and if each particle is equivalent to each cell, there is a 10E6 economy of scale by producing particles to replace the current successes in cell-based modified tumor biology approach for cancer therapy.

### SUMMARY OF THE INVENTION

This invention provides for the formation, production, and in vivo delivery of recombinant molecules for therapeutic purposes. The invention could contain one or more than one molecule or contain native cell surface components from a particular cell type. Molecules preferably include any amino acid moiety-containing molecule, but other molecules captured during the process covered by this invention could be envisioned. Formation of specific molecules could be engineered genetically by molecular biology techniques to be expressed on the surface of cells that alone or together with native molecules on the said cells' surface, forms the essence of the invention. The formation and production of the invention involves the removal of cell surface membrane components as a consequence to the budding of particles from cells. The particles could be made of single or multiple components. Components are envisioned to be viral in origin, but could be induced by non-viral methods, or natural to the cell selected host. The invention is preferably for in vivo delivery, but could be used in vitro for induction or maintenance of cellular processes. Processes include, but not limited to, cellular signaling, cellular induction, cellular suppression and/or cellular attractant. In vivo delivery could be by intravenous injection, but other routes include but are not limited to oral, suppository, intra-muscular, inter-cranial, inter-peritoneal, or directly into mammalian organs, capillaries, ducts, or lymphoid system either alone or associated with biological or non-biological materials and/or devices. Inter-respiratory devices, cutaneous, and topical applications are also envisioned within this invention. In addition the application of the invention as aerosols, creams, puffers, or on surfaces, is included in this invention. Surfaces include, but not limited to, synthetic, non-synthetic, biological, or non-biological matrixes including autologous, allogeneic, and xenogeneic extracellular matrix materials. Therapeutic purposes encompass all procedures and/or processes that result in the improvement or intended improvement of the health and well being of an inflicted human or mammalian host.

This invention using a biological particle/carrier approach provides several advantages that would dramatically affect the outcome of vaccination. These parameters include:

(1) Ability to provide significant amount of MHC tumor peptide (signal 1) within an optimal immunologic context—co-stimulatory molecules for signal 2—to provoke an effective anti-tumor immune response. In all preclinical models in which vaccine cell numbers were evaluated, increasing the number of vaccinating cells increase the potency of systemic immunity. For this purpose, the substitution of cells for particles may allow the delivery of more immunogenic material at the site of vaccination without requiring tumor cell expansion.
(2) Biological particles/carriers would allow a poly-epitopic presentation that would avoid tumor antigen and MHC variant loss following vaccination with a single epitope.
(3) Expression level of ligand/mediator containing particles could be engineered within established MHC-matched or possibility MHC-unmatched cell lines and released criteria established and tested in vitro to determine the ability of the preparation to induce immune responses. Established cell lines lend consistency between product batch production lots.
(4) Route of administration of the vaccine that is most effective at generating an effective anti-tumor immune response. As mentioned in the first parameter, cell dose is often limited in clinical testing related to the amount of material available to make the vaccine. For cell-based therapies, preclinical models are difficult to completely analyze routes of injection since the dermis of rodents is too thin to place large numbers of cells. This will not be the case for biological particles/carriers where particles are the size of viruses and are amendable to concentration of 100 to >1,000-fold, requiring tens to hundreds of microliters for dose delivery of >10E7 cell dose equivalence. However, the optimal route of administration may differ for different histological types of tumors, and therefore, additional routes of particle delivery are possible within a mammalian host. The biological particles/carriers technology lends itself to all types of delivery options, including aerosol and suppository administration.
(5) Ability of re-infused ex vivo generated dendritic cells to traffic intact to sites of antigen presentation—the spleen and draining lymph nodes—is limited, and those that do traffic appropriately are often cleared by host cytotoxic T-lymphocyte (CTL) activity. The carrier technology lends itself well to trafficking and evading clearance from the body while still maintaining immune stimulatory activity. Viruses are well known to navigate throughout the body without detection.
(6) Safety—the biological particles/carriers are not infectious, intended not to contain any genetic material. The viral-like components behave as a scaffold where appropriately configured protein molecular complexes are embedded.

Cancer can effect any organ in the human body, including breast, with 190,000 new cases reported each year; prostate with 180,000 cases diagnosed and treated annually with 32,000 annual deaths; lung with 175,000; colon with more than 94,000; head and neck with 70,000; gynecologic malignancies with more than 82,000; bladder with 55,000; pancreatic with 26,000; kidney with 31,000; brain with 17,900; sarcomas with 8,900 cases diagnosed on an annual basis, occurring in children and adults. The present invention relates to treatments of these cancers, but not limited to these types. Current therapies include surgery, radiation therapy, chemotherapy, and bone marrow transplantation. New treatment approaches are needed to reduce costs. The biological particles/carriers technology could substantially improve and/or compliment current cancer therapies by using universal appropriately modified tumors as host cells to provide particles that will be the biopharmaceutical basis for treatment.

In addition, the present invention will simplify the manufacturing and production process for cancer vaccines. The technological innovation of this invention is that an established cell-mediated anti-tumor effect is reduced and simplified to a particle-mediated anti-tumor effect. The product is envisioned to be a lyophilized preparation stable at room temperature storage that has an economy-of-scale production and manufacturing advantage since each cell produces tens of millions of gag particles, each capable of inducing a T-cell response. The particles have no intrinsic activity. They are neither infectious nor self-replication competent, and are not intended to contain any nucleic acids. They are intended to mediate responses through cell surface interactions, triggering signal-transduction events upon engagement of cognate receptors on T cells in vivo, but could be envisioned as foreign bodies that could be taken up by other cell types for presentation to the host immune system. The commercial application is in the area of cancer where the product would greatly improve the availability of anti-tumor technology in a clinical setting.

In one aspect of the invention the host cell producing the biological particles/carriers will be an established tumor cell line. This cell line will have MHC molecules present on the cell's surface loaded with pre-processed antigens specific to that tumor. Co-stimulatory molecules could be engineered into the established tumor cell line by the introduction of specific cDNA by mechanical, physical, chemical, or viral means. Mechanical, physical, and chemical means include but not limited to electroporation, and/or lipid-mediated, polyethylene glycol, Sendai virus membrane fusion that bypasses the cellular membrane to gain access to the cellular chromatin structure where integration may or may not occur. Viral mediated delivery mechanisms include but not limited to murine leukemia virus (MuLV), adenovirus, adeno-associated virus (AAV), lentivirus, and canarypox vectors. In another embodiment of this aspect, the host cell for the biological particles/carriers could be primary cells derived from the tumor. In another embodiment of this aspect, the host cell for the biological particles/carriers could be a transformed cell line. In still another embodiment of this aspect, the host cell for the biological particles/carriers could be a cell line not related to the tumor, but a universal cell line expressing specific (one or more) tumor antigens in the presence of co-stimulatory molecules. In all embodiments of this aspect, the host cell for the biological particles/carriers could be autologous, allogeneic, or xenogeneic with respect to the intended mammalian recipient of the therapy.

In all aspects of the invention the particle released from the host cells generating the biological particles/carriers is non-infectious. In one embodiment the biological particles/carriers production could be innate to or induced by the introduction into the host cell of viral or non-viral components by mechanical, chemical, and/or viral vector means. In another embodiment the biological particles/carriers production from the host cell could be due to the expression of one or more viral matrix proteins, for example, but not limited to, HIV-1 gag protein or the M1 matrix protein of the Influenza virus. In still another embodiment the biological particles/carriers released from the host cell could be an infectious viral particle that is later inactivated by various chemical means including, but not limited to nucleic acid crosslinking inactivation. In all embodiments the released particles could be harvested; concentrated by various methods, including, but not limited to polyethylene glycol; and lyophilized for long-term storage prior to therapeutic use in vivo.

The present invention describes an immune stimulation technology that has demonstrated the ability to stimulate T cells by incorporating over-expressed cell surface co-stimulatory proteins into either active or inactive viral particles and/or virus-like-particles. The process relies on the biological process of particle release to remove pieces of the cellular membrane while exiting a said host cell. Host cells are modified to release virus-like-particles or infectious virus particles that are subsequently inactivated together with recombinant co-stimulatory proteins displayed onto the surface of cells by standard molecular biological transfection and/or transduction techniques. The recombinant co-stimulatory proteins include, but not limited to CD40, CD40 ligand, CD30, CD30 ligand, 4-IBB receptor or ligand, CD27, FAS receptor or ligand, and TRAIL receptor or ligand. In fact, the recombinant protein or proteins that mediate the anti-tumor effect could be a cytokine or antibody that either directly or through accessory cells induce an immune response against the tumor. As a cytokine, the molecule could be, but not limited to an interleukin (IL-2, IL-12, IL-15, IL-23), a colony stimulatory factor (GM-CSF) or tumor necrosis factor (TNF-alpha). As an antibody, the molecular could be, but not limited to a T or B cell receptor component (anti-CD3 or anti-CD20); a co-stimulatory receptor (anti-CD28); or an activation modulatory molecule (anti-CTLA4). In fact, the anti-tumor response could be due to apoptosis of immune reactive cells as could be the case in autoimmune diseases. As an example, but not limited to the disease—myasthenia gravis, particles containing an auto-reactive antigen in the presence of an apoptotic molecule could delete reactive immune cells. In all cases, the released particles contain the same over-expressed protein present on the host cells' surface and as such, serve as a novel delivery system for recombinant molecule signaling. In this way, single or multiple molecules are expressed with similar native structure to the naturally expressed human or mammalian protein.

The “capture” of a protein on the surface of a particle simplifies the process of synthesizing and purifying recombinant molecules and/or proteins to harvesting virus particles. Thus, in vitro recombinant protein systems can be simplified to purification of viral/non-viral/cellular particles or viral-like-particles using standard generic techniques. At the same time this technology insures proper orientation, conformation, and post-translational modifications of the synthesized protein, since the protein is made de novo.

In summary, the present invention describes the utility of a process to in vivo deliver immune modulator signals to a host immune system in the area of cancer biology, resulting in anti-tumor responses. The invention has applications to a wide range of tumors, including but not limited to direct presentation of specific tumor antigens in the context of MHC presentation with co-stimulatory signaling to T-lymphocytes. The invention mimics antigen-presenting cells with a similar efficiency as dendritic cells to present antigens to the immune system. The invention could also serve as a source of tumor antigen for uptake by host antigen-presenting cells, which in turn could present the tumor antigens to the host immune system. The invention provides a method to produce large amounts of material with efficacy similar to cell-based therapeutic approaches. The invention is a biological agent expressed as a particle containing one or more recombinant protein for in vivo use to induce, modify, and enhance immune cellular processes.

### BRIEF DESCRIPTION OF THE DRAWINGS

The invention is further described by the accompanying drawings and the description thereof herein, although neither is a limitation of the scope of the invention. Although the biological particle/carrier system has not been tested in the area of tumor biology, it has been tested and shown to induce T cell proliferation. T-cell stimulation is a requirement for immune recognition and immune stimulation, resulting in anti-tumor responses.

FIG. 1 is a schematic representation of constructing a virus-like particle producing tumor cell line. The figure illustrates the introduction by electroporation of plasmid vectors expressing a particle budding system. The particle budding system could be produced by a number of different mechanisms; as examples, the expression of the HIV-1 gag protein or the M1 matrix protein from the Influenza virus is shown. In this embodiment, a tumor cell line would be continuously processing cellular proteins and displaying the processed peptides in the context of class I and class II MHC molecules. Some of the processed cellular proteins would be tumor specific and in the presence of co-stimulatory molecules could induce immune reactions against that specific type of tumor. The introduction of the particle budding system into the tumor cell would release virus-like particles where the molecules present on the surface of the host tumor are incorporated into the particle. The end result of such modifications of the tumor cell is that the release particles can substitute for the tumor cells.

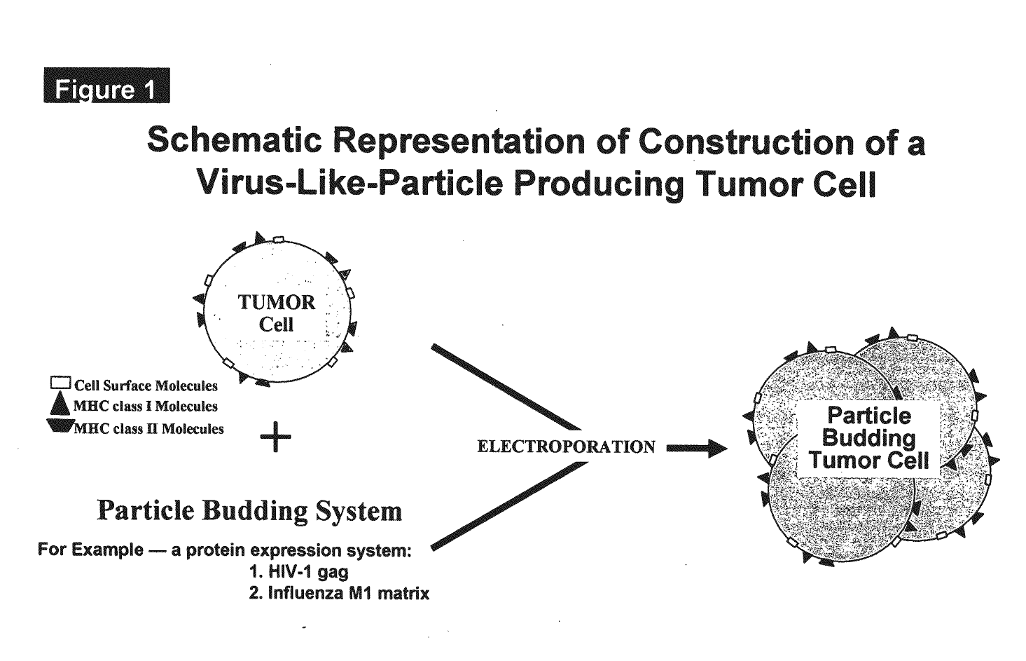

FIG. 2 is a schematic representation of constructing a virus-like particle containing co-stimulatory molecules. The figure illustrates the introduction, by retroviral vectors, of particles containing co-stimulatory molecules. In this embodiment, B7 family co-stimulatory molecules are expressed on to the surface of tumor cells already expressing the particle budding system. As the particles are released from the tumor cells they would incorporate, in addition to MHC molecules containing processed peptides, co-stimulatory molecules. The introduction of co-stimulatory signaling molecules into the tumor cells along with MHC associated processed tumor antigens would impart onto the released particles antigen presenting capabilities. These capabilities could mimic cells present within the mammalian immune system to result in “dendritic-like” cells capable of anti-tumor responses.

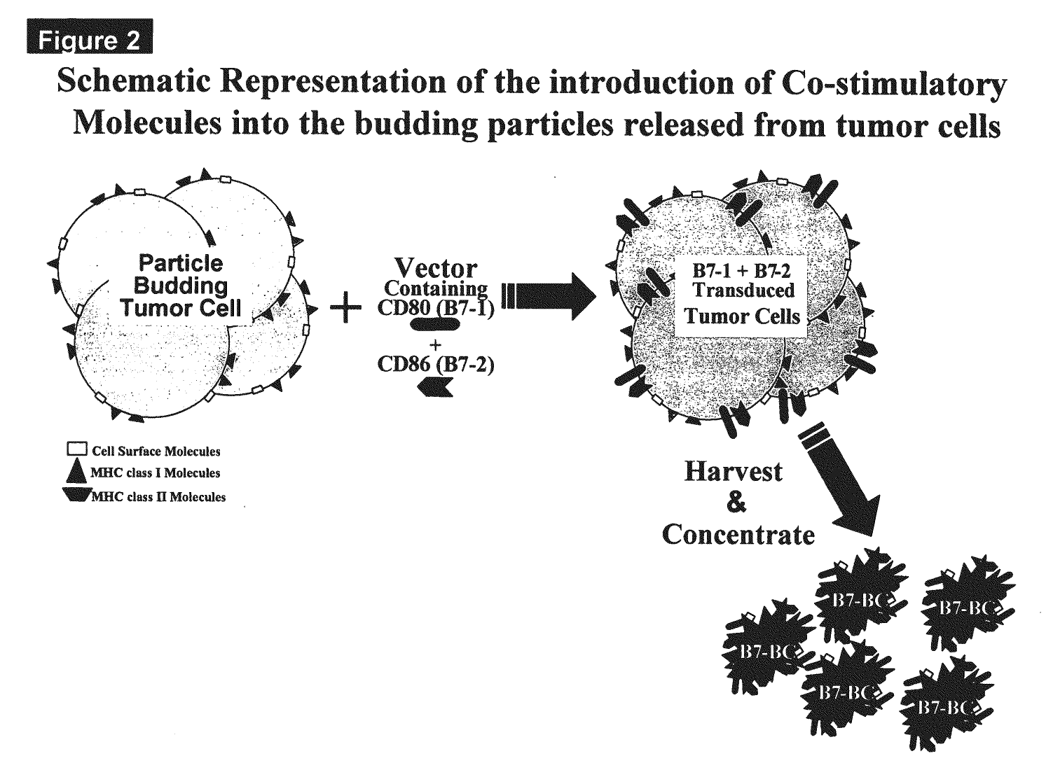

FIG. 3 identified the inability of biological particles/carriers released from viral infected host cell lines to induce T cell proliferation and activation. Two types of biological particles/carriers were made. The host cell used in the case of herpes simplex virus was, Lof(11-10), a fibroblastic line capable of infection by herpesviruses (HSV). The host cell used in the case of human immunodeficiency virus (HIV) was a chronic T cell line, A3.01, continuously expressing HIV particles. The viral-specific biological particles/carriers were collected from the supernatant of host cell cultures and inactivated with a UV-activated DNA crosslinker. The preparations were non-infectious by the lack of p24 release from exposed CD4+ T cells (HIV) or lack of cytopathic cell lyses in susceptible fibroblasts (HSV-2). The experiment shown used human peripheral blood mononuclear cells (hPBMCs) from 5 different healthy donors. The hPBMCs were exposed to the biological particle/carrier preparations at the start of culture, time points were taken (only 1 shown), and proliferation assays perform using AlamarBlue™. The biological particle/carrier preparations were derived from either unmodified host cells. For the three hPBMC donor cells tested, the degree of T-cell proliferation upon exposure to biological particle/carrier preparations derived from unmodified cells was equal to that observed in untreated hPBMCs and not to that observed in PHA-stimulated cultures, demonstrating that the released native particles are not intrinsically immune stimulatory.

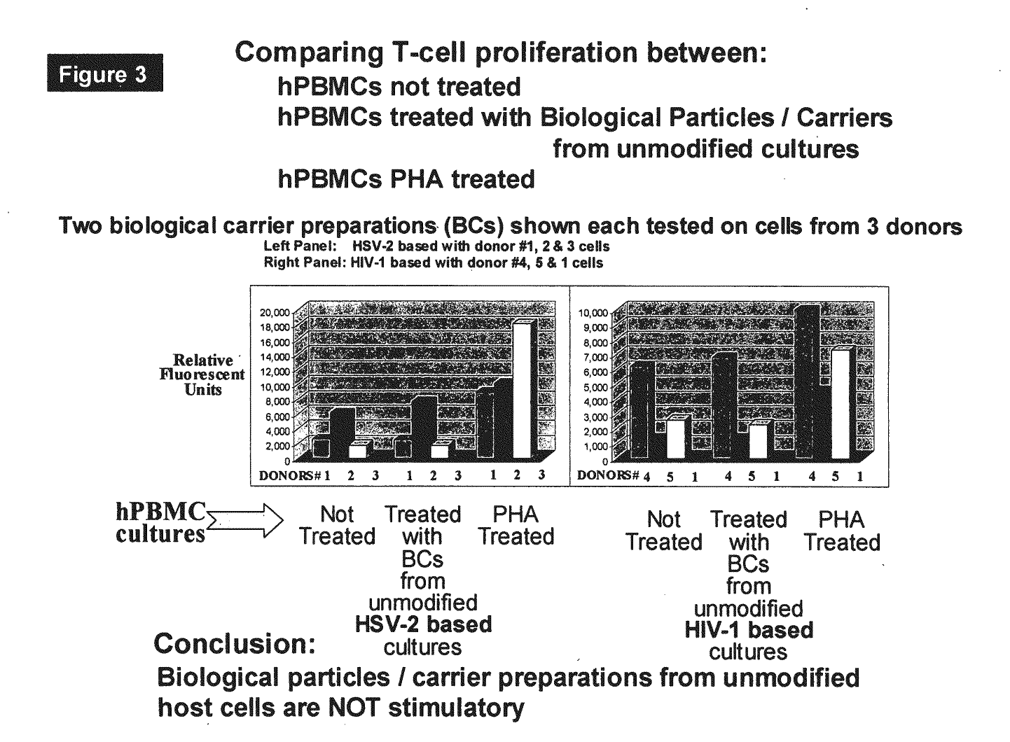

FIG. 4 identified the ability of biological particles/carriers released from co-stimulatory modified viral infected host cell lines to induce T cell proliferation and activation. The same 2 types of biological particles/carriers made in FIG. 3 were used here. The hPBMCs were exposed to biological particle/carrier preparations derived from either unmodified or co-stimulatory modified host cells. For the three hPBMC donor cells tested, the degree of T-cell proliferation upon exposure to biological particles/carriers (either HSV- or HIV-based) derived from co-stimulatory modified cells were stimulatory. Whereas the hPBMC cultures exposed to biological particles/carriers from unmodified host cells were not, similar to that shown in FIG. 3. Co-incubation experiments with host cells and hPBMCs showed that only the B7-modified host cells stimulated T-cells. The data supports the notion that released particles retain the stimulatory activity of the host cells and the addition of B7 to the host cells resulted in particles that could stimulate T cell proliferation.

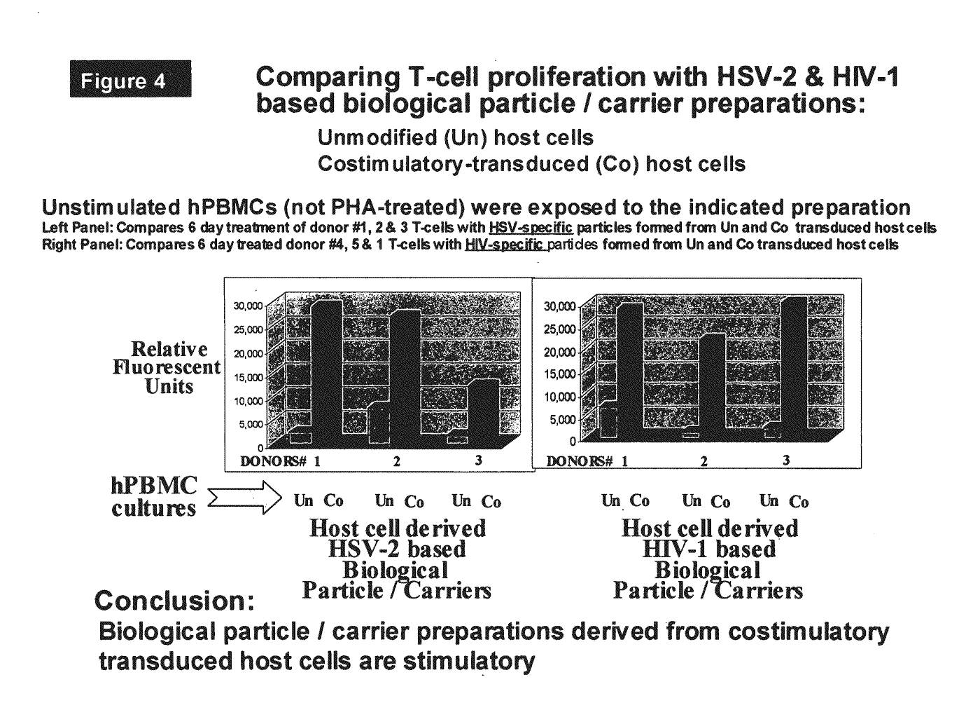

### DETAILED DESCRIPTION OF THE PREFERRED EMBODIMENTS

The present invention relates to the use of particles to capture and incorporate surface molecules that are displayed naturally and/or purposely expressed on the surface of host cells by recombinant molecular biologic techniques. The naturally displayed molecules could be tumor—derived processed antigens associated with MHC molecules or molecules that assist in presenting processed antigens to the immune system. The assisting molecules could be from the class of molecules known as co-stimulatory molecules that consist of surface expressed molecules (B7 family members, members of the TNF family, and/or other immunoglobulin family members—ICAM and VCAM), cytokines (interleukins and lymphokines), and/or fatty acids (prostaglandin). The purposely expressed molecules could be tumor—derived antigens or assisting molecules detailed above that help facilitate immune responses to processed tumor antigens. The generated particles are released from said host cells either naturally as the result of a viral infection or preferably by the introduction of a particle generating system. The particle generating system is established within the host cell by either the permanent or transient expression of one or more viral protein components that are capable of generating released viral-like particles that are not infectious and contain no purposely incorporated nucleic acid. The invention describes a process and the utility of that process to develop therapeutic entities that can modulate cellular processes that can protect against cancer by suppressing tumor formation.

In the preferred embodiment, the particles used in the invention are produced in vitro from cells genetically engineered to express recombinant molecules onto their cells' surface and genetically engineered to produce budding particles that capture and incorporate these expressed recombinant molecules such that the particles when harvested contain the recombinant molecules. In accordance with the invention, the particles are virus-like-particles and as such are not infectious, but rather serve as biological carriers of expressed recombinant molecules that are removed from the cells' surface as the particle is released from the cell. Such particles could be harvested and then used as recombinant molecules in vitro and in vivo in accordance with the invention to induce an anti-tumor environment within a mammalian host.

The invention provides for the use of the recombinant molecule(s) containing particles to present the relevant molecule(s) to the immune system. Pursuant to the present invention, molecules have been expressed and/or induced on the surface of the continuously expressing particle-producing host cell line, and the released particles are harvested. The recovered particles present transduced or endogenously expressed antigen(s) together with co-stimulatory molecule(s) directly to the immune system, or are picked up by “professional” antigen presenting cells (APCs), such as dendritic cells and macrophages, for presentation to lymphoid cells. The minimum requirement of an APC for activation of T-lymphocytes are to degrade complex protein antigens into antigen fragments, to present these antigen fragments that were bound to MHC molecules present on the particles by virtue of their presence on the host cell surface and subsequently captured and incorporated into particles along with the recombinant expressed co-stimulatory molecules, like B7.1 and B7.2.

Techniques and terms for transduction, sequence isolation, in-frame fusions or ligation, gene, recombinant, coding of sequences, intracellular-transmembrane domains, ecto- or extracellular portions of proteins, genetically-modified, virus-like-particles, virus infection, inactivation of virus particle preparations, viral budding, digestion or restriction enzyme analyses, in addition to other molecular biologic or molecular virology techniques and terms that are established and used in the art are described in standard laboratory manuals and references, such as, for instance, Sambrook et al., MOLECULAR CLONING, A LABORATORY MANUAL, 4^{nd} Ed.; Cold Spring Harbor Laboratory Press, Cold Spring Harbor, N.Y.

Tumors against which the present invention may be applicable in the formation of biological particles/carriers containing anti-tumor activity include cancers that can effect any organ in a mammalian host, including humans, but are not limited to breast; prostate; lung; colon; head and neck; gynecologic malignancies; bladder; pancreatic; kidney; brain; sarcomas, and tumors from mesenchymal, endodermal, or ectodermal origin.

Antigens against which the present invention may be applicable in the formation of particles containing recombinant forms include polypeptides/lipids encoded by the tumors listed above. The multitudes of antigens encoded by these agents that may be expressed include, but are not limited to external surface proteins and structural proteins including enzymes, transcription factors, and other cell regulatory proteins. Proteins include all known and to be discovered gene or nucleic acid containing encoded proteins, cytokines and related molecules such as interleukins, growth factors, chemokines, adhesion molecules, neurotrophic factors, MMPs/TIMPs, receptors, and developmental proteins. Peptides include any amino acid sequence that could be made and/or found in nature; expressed as monomers or as oligomeric versions, including immune-dominant epitopes. Two types of antigens have been identified on tumor cells: Tumor-specific transplantation antigens (TSTAs) that are unique to cancer cells, and tumor-associated transplantation antigens (TATAs) that are found on both cancer and normal cells. Thus, tumor antigens consist of TSTAs, TATAs, and oncogene proteins. Tumor-specific antigens have been identified on tumors induced by chemical and physical carcinogens and some virally induced tumors. The antigen(s) can be present within the host tumor cell(s) that is used as the particle-producing host or as part of a transduction and/or transfection process by biological (viral vectors), chemical (liposomes), or mechanical (electroporation) methods.

The following examples further illustrate experiments that have demonstrated reduction to practice and utility of selected preferred embodiments of the present invention, although they are in no way a limitation of the teachings or disclosure of the present invention as set forth herein.

### Example 1

Genetic construction of modified human tumor cells—gag-expression vector with and without B7-DC surface expression. Experience from the HIV- & HSV-based biological particles/carrier preparations demonstrate the need to obtain high expression of released particles. This was especially true for the HIV-based particles where only preparations made from chronic-infected cells expressing a certain level of p24 activity were active in T cell stimulation. Once a line was engineered to express that level of p24, all preparations made from that line were active. As a result, the HIV-based line we constructed could generate microgram amounts of p24 antigen protein per milliliter of culture supernatant. In order to obtain a similar elevated level of p24 expression, we propose to construct an HIV-gag expression system dependent on HIV-tat transactivation of the HIV-LTR in the absence of HIV-env so that only virus-like particles composed of the gag protein would be elaborated into the culture. HIV-gag like many other viral core proteins, Influenza M1 matrix protein for example, is capable of budding from cell surfaces without the assistance of other viral coded proteins. This provides a mechanism by which the biological particle/carrier technology can be performed with non-infectious virus particles, rather than infectious particles that required UV chemical inactivation before use. Non-infectious viral-like particles add a level of safety to the biological particle/carrier system. The main reason for HIV-gag rather than Influenza-M1 is that commercial kits are available to measure HIV-gag p24 antigen, which are not available for other matrix proteins. Because of the toxicity associated with various HIV-encoded gene products, we will use a minus tetracycline-dependent expressed HIV-tat gene system, where tat will be expressed only upon the removal of the drug tetracycline from the culture system. This tat expression system (pBI-tat-tTA, not shown) will be co-transfected with the HIV-gag/rev/RRE, minus env containing expression system (pAG131, not shown) along with a CMV-puromycin gene as a selectable marker by either electroporation or using SuperFect™ reagent. The pAG131 expression system contains all the HIV gene components (rev and the rev-response-element, RRE) that will allow efficient expression of the HIV-gag protein without assembly of infectious virus due to the absence of the HIV-env gene gp160. Puromycin resistance will be used to select transfected cells followed by flow cytometry sorting for GFP high cells upon removal of the drug tetracycline from the cultures. The GFP gene is expressed on the same mRNA transcript as tat and its ability to fluoresce in the FITC channel after excitation will be used as a secondary selection for the highest producing p24 antigen cells. The actual p24 activity will be quantified by commercial HIV p24 antigen capture ELISA kits. The transfectants that illustrate the highest p24 activity after removal of tetracycline from the culture will be further induced with TPA and TNF. The addition of 12-o-tetra-decanoyl phorbol-13-acetate—TPA and the cytokine tumor necrosis factor—TNF is known to activate the HIV-LTR by interacting with the two NFκB binding sites to further increase gag expression 10- to 100-fold.

Once the human tumor cell lines contain the gag expression system and demonstrate the ability to generate microgram amounts of p24 per milliliter of culture supernatant, the cell lines will be once again engineered to express one or more co-stimulatory surface molecules. Similar to the gag/tat/puromycin vector transfection, the co-stimulatory surface molecule(s) containing expression vector will be co-transfected with a SV₂-neomycin gene as a selectable marker by either electroporation or using SuperFect™ reagent. Secondary G418 selection of these transfected cell lines will result in human tumor cells containing both gag and co-stimulatory molecule(s) expression with resistance to the combination of puromycin and the G418 drugs.

### Example 2

A Universal cell line expressing specific tumor antigens and co-stimulatory molecules. Although using cells from a specific tumor will result in a poly-epitopic presentation, in the case where a specific tumor antigen has been shown to be efficacious, vaccination with individual epitopes may be more desirable. In this case, the host cell generating the biological particles/carriers could be a non-tumor cell or a cell from a different tumor—a universal cell—that could be modified to express on its surface tumor specific antigens in a similar fashion to the surface expressed co-stimulatory molecules. The release of particles from a universal cells, such as, but not limited to a spontaneous transformed CD4 positive cell line—A3.01, will incorporate said tumor antigen(s) and co-stimulatory molecule(s) as the particle exits the surface modified A3.01 cell line.

### Example 3

Formulation of biological particles/carriers for use as an anti-tumor therapeutic. Cultures of genetically modified cells releasing biological particles/carriers will be expanded to grow in multi-stack factories for adherent cells or roller bottles for suspension cells. Induction protocol similar to that described in Example 1 will be used to maximize the particle release and harvest. After the induction procedure, the culture fluid would be collected and clarified by centrifugation at 4,000 rpms for 20 minutes in 1 liter bottles and polyethylene glycol (PEG) added (6 to 15%), mixed, and stored at 4° C. overnight. The following day, the precipitated material would be collected by centrifugation and resuspended in buffer, aliquots made, lyophilized, and stored at 4° C. as a 1× to 1,000× concentrated preparation. The amount of biologically active preparation in the individual aliquots would be adjusted for a therapeutic dosage. If necessary, the particle preparations would be further purified using techniques of ultra-centrifugation, filtration, and/or chromatography.

### Example 4

Monitoring the effectiveness within an animal model of a biological particle/carrier preparation compared to whole cell-based therapy. The present state of the art in anti-tumor biological therapy is cell-based modification of “killed” tumor and/or by-stander cells. One of the most successful approaches in cell-based cancer immune-therapy is GM-CSF expression of implanted cells. Experiments could be conducted to compare the ability of irradiated GM-CSF modified tumor cells to inhibit tumor formation compared to biological particle/carrier preparations generated from co-stimulatory modified tumor cells. Mouse models have longed been used to test efficacy of anti-tumor approaches; in fact, successful anti-tumor results from implanted GM-CSF modified tumors/by-stander cells in mouse models have lead to human clinical trials in human. This example in a mouse model demonstrates reduction to practice and utility of the invention, detailing comparative testing between implantation of modified host cells versa implantation of biological particles/carriers. The model lymphoma B cell tumor line—A20HA—described in this example uses a model antigen—Influenza hemagglutinin (HA) gene—transfected into the murine A20 tumor cell line. The expression of the HA antigen on the tumor allows monitoring of the activation of HA-specific CD4+ T cells isolated from T-cell receptor transgenic mice in vitro. Using this model antigen, experiments can be conducted to compare cell-based versa particle-based stimulation of HA-specific T-cells.

Proliferation assays will be performed where either irradiated unmodified A20HA cells or mock released particles (negative control); irradiated GM-CSF modified A20HA cells or mock released particles (control); irradiated gag modified A20HA cells or released particles (test #1); and irradiated gag+B7-DC modified A20HA cells or released particles (test #2) will be co-incubate with fresh splenocytes obtained from either non-transgenic B10.D2 mice, HA-specific CD4 T cell receptor transgenic mice 6.5, and HA-specific CD8 T cell receptor transgenic mice Clone 4 (8×10E4/well). Biological particles/carriers preparations will need to be titrated to determine a dose response curve; the preparations will be quantified by the amount of gag protein present. We would anticipate gag concentrations to range from 1 ng to 1 ug in the assay. The cells will be pulsed with ³H-thymidine (1 mCi/well) after 3 days in culture. Cells will be harvested 18 hours later with a cell harvester. ³H-thymidine incorporation into DNA will be measured as counts per minute (cpm) on a direct beta counter. Data will be calculated as cpm in the test groups minus cpm from unmodified group divided by the number of clonotype-positive cells in the well as determine by flow cytometry. Values will be displayed as the mean+/−SE cpm/100 clonotype positive T cells per well.

As a correlate to the proliferation data, at 48 hours, supernatants will be collected from the plates used in the proliferation assay and stored at −70° C. until assayed for 1L-2 and IFN-gamma by ELISA. Data represent mean+/−SE of triplicate cultures from each group and are expressed as the amount of cytokine produced per 100 clonotype positive T cells.

To assess the ability of B-lymphoma cells to present antigen to naive splenocytes from non-transgenic and HA-specific transgenic mice in vitro, irradiated (10,000 rads) A20HA unmodified and modified cells (1×10E5 cells/well) could be mixed with 5×10E4 splenocytes. After 24 hours, supernatants could be collected and assayed for IL-2 and TN-gamma by ELISA.

### Example 5

Ability of biological particle/carrier preparations to inhibit tumor cell growth. Additional experiments could be done to test the ability of co-injected particles to inhibit the growth of live non-irradiated A20 tumor cells (a murine B cell lymphoma tumor line). A20 cells will be titrated (10E4 to 10E6) and co-injected subcutaneously into the hind leg of BALB/c mice with particle preparations obtained from modified A20 cells; gag modified A20 cells; and gag+B7-DC modified A20 cells; in addition to, irradiated GM-CSF modified A20 cells as a positive control. Titration (0.1 ug to 100 ug with respect to gag) of the biological particle preparations will be done to determine the amount required to inhibit tumor growth.

Since previous studies showed that intravenous injection of A20 vaccines did not induce systemic immunity, BALB/c mice will be vaccinated subcutaneously, but if the subcutaneous results are positive, intravenous inoculations in subsequent experiments will be tested. Additionally, other forms of tumors could be tested in vitro (for T cell specific HA responses) and in vivo (for tumor inhibition), including a renal cell carcinoma—RENCA/RENCA-HA tumor cell model system.

### Example 6

Pre-immunization prior to tumor challenge. Mice could be vaccinated twice with biological particles/carriers generated from either unmodified tumors cells; gag modified tumor cells; gag+B7-DC modified tumor cells; in addition to, irradiated GM-CSF modified A20 cells as a positive control. Both in vitro and in vivo titration experiments using the particle preparations will be used to determine the appropriate amount of biological particle/carrier preparation to use in these vaccinations.

One week after the second vaccination, mice will be challenged intravenously with live, non-irradiated tumor cells. A titration of tumor cell number (10E4 to 10E6) will be tested to assess the efficiency of the vaccination in mice. Note that the intravenous administration of tumor cells will allow further quantification of tumor growth within specific organs.

### Example 7

Therapeutic testing of biological particles/carriers. Five and eleven days after intravenous injected of mice with 10E5 live non-irradiated tumor cells, biological particles/carriers generated from either unmodified tumor cells; gag modified tumor cells; gag+B7-DC modified tumor cells; in addition to, irradiated GM-CSF modified tumor cells as a positive control will be injected subcutaneously. The concentration of biological carriers to be used will be equal to or greater than that shown most effective in the above example experiments. Mice will be followed daily for survival.

This example relates to the incorporation of immune modulator molecules into cell lines that express particles that capture and incorporates said molecules. These immune molecules could include one or more of the following proteins, but are not limited to these molecules—B7.1, B7.2, CTLA-4, OX-40, CD27—that are involved in the activation or suppression of immune responses. In addition to these molecules in some situation, specific antigens to cancerous tumors would be included into the release particles by their inclusion onto the host cells' surface. In addition to the incorporation of immune and antigen molecules that were exogenously expressed on the cells' surface by standard molecular biological techniques, native cellular expressed molecules are expected to be co-incorporated into the released particles. These molecules would include processed peptides from the exogenously expressed antigens within the groove of MHC class I and class II, plus CDI molecules. The processed peptides and glycolipids associated with MHC and CD1 molecules, respectively, would stimulate immune responses by binding to the CD3 molecule and the T-cell receptors of appropriate cells, while the immune modulator molecules will interact through there respective ligands or receptors. Although the mechanism of these approaches might be induction or repression of immune responses through humoral and cell-mediated arms of the immune system, other mechanisms may be implored to affect immune modulation that may involve but not limited to the expression of cellular factors that influence immune responses.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth.

1. A process to induce an effector cell mediated immune response against tumor cells in a cancer patient, said method comprising
administering, to a cancer patient, a non-infectious, biologically generated virus particle with a cellular membrane from a host cell, said membrane comprising
an MHC molecule that presents one or more tumor specific antigens, and
a co-stimulatory molecule,
wherein said non-infectious virus particle is an inactivated intact virus particle that was infectious and
wherein said administering is of an amount effective to induce an effector cell mediated immune response against tumor cells in said patient.

2. The process of claim 1 wherein said immune response is mediated by T cells.

3. The process of claim 1 wherein said non-infectious particle is released from a homologous tumor cell from the patient.

4. The process of claim 1 wherein said non-infectious particle is released from a matched major histocompatibility complex containing tumor cell.

5. The process of claim 1 wherein said non-infectious particle is released from a non-homologous tumor cell line containing one or more matched human leukocyte antigens.

6. The process of claim 1 wherein said immune response reduces the number of tumor cells in said patient and thereby treats cancer in said patient.

7. The process of claim 1 wherein said host cell expresses one or more tumor specific antigens on the cell's cell membrane and said particle has a membrane that further comprises the one or more antigens.

8. The process of claim 1 wherein said particle mimics a dendritic cell.

9. The process of claim 1 wherein said host cell is a non-tumor cell.

10. The process of claim 1 wherein said co-stimulatory molecule is a B7 family molecule.

11. The process of claim 1, wherein said co-stimulatory molecule is selected from CD40, CD40 ligand, CD30, CD30 ligand, 4-IBB receptor, 4-IBB ligand, CD27, FAS receptor, FAS ligand, TRAIL receptor, and TRAIL ligand.

12. The process of claim 1, wherein said co-stimulatory molecule is a cytokine.

13. The process of claim 12, wherein said cytokine is selected from an interleukin, a colony stimulatory factor, and a tumor necrosis factor.

14. The process of claim 13, wherein said interleukin is selected from IL-2, IL-12, IL-15, and IL-23.

15. The process of claim 13, wherein said colony stimulatory factor is GM-CSF.

16. The process of claim 13, wherein said tumor necrosis factor is TNF-alpha.

17. The process of claim 1, wherein said co-stimulatory molecule is an antibody.

18. The process of claim 17, wherein said antibody is selected from anti-CD3, anti-CD20, anti-CD28, and anti-CTLA4.

19. The process of claim 1, wherein after said administering, the exogenous tumor specific antigen is transferred to an antigen-presenting cell in said cancer patient, and the antigen-presenting cell induces the effector cell mediated immune response against tumor cells in said cancer patient.

20. The process of claim 1, wherein the exogenous tumor specific antigen is a tumor-specific transplantation antigen (TSTA).
